(19) United States
(12) Patent Application Publication
(10) Pub. No.: US 2004/0092819 A1
(43) Pub. Date: May 13, 2004

(54) Method for treating laminitis in horses, ponies and other equines with acoustic waves

(75) Inventor: Roderick Richards, Winterthur (CH)

Correspondence Address: HMTAG C/O STEFFEN STRAUSS, KREUZLIUGERSTR. 5, LEUGWIL, CH-8574, CH

(73) Assignee: HMT High Medical Technologies AG, Lengwil (CH)

(21) Appl. No.: 10/636,665

(22) Filed: Aug. 8, 2003

Related U.S. Application Data
(60) Provisional application No. 60/403,746, filed on Aug. 16, 2002.

(51) Int. Cl. A61B 8/00

(52) U.S. Cl. 600/437

(57) ABSTRACT

This invention relates to a method for veterinary medical treatment of Laminitis in Horses, Ponies and other Equines with acoustic shock waves. More particularly, the invention relates to a method for using acoustic shock waves to normalize the blood flow within the laminae and thereby reverse the laminitic process.

### BACKGROUND OF THE INVENTION

1. Field of the Invention

This invention relates to a method for veterinary medical treatment of Laminitis in Horses, Ponies and other Equines with acoustic shock waves. More particularly, the invention relates to a method for using acoustic shock waves to normalize the blood flow within the laminae and thereby reverse the laminitic process.

2. Description of the Problem

Laminitis is a disease which can strike any hoofed animal and ungulates with a very high morbidity and frequent mortality rate. But it is primarily known to affect equine. Laminitis, is a potentially devastating condition which often spells the end of a horse's career. It used to be a death sentence for the horse that contracted it.

The disease is caused by one or a combination of various factors such as mechanical stress, systemic disease, psychological stress and or dietary components. Laminitis is a serious threat to the horse in that it generally causes changes which lead to a reduction or total lack of use. In larger breeds it often leads to euthanasia.

The disease manifests itself in acute lameness usually of the fore feet but may include the hind feet. The laminae make up the structure responsible for anchoring the coffin bone to the hoof wall. The laminae consist of an arrangement of non-sensitive laminae made of keratin and sensitive laminae that contain blood vessels and nerves. Both types are connected together in a dovetail fashion that suspend the coffin bone within the hoof.

The hoof of the equines is composed of a hard hoof composed of dense keratinised fibers. The distal phalanx of the digit is effectively suspended within this structure and attached to the growing hoof by a large number of microscopic interdigitating laminae. These structures have a highly complex and poorly understood system of arterio-venous anastamoses which regulate the blood supply to the lamina to compensate for various physiological states and mechanical stress.

Inflammation of these structures is caused when the control of the arterio-venous anastamoses is disturbed leading to a compartment syndrome where the blood flow and hence nutrient and oxygen supply to the lamina is compromised. This leads to mechanical tearing or separation of the lamina which ultimately leads to the collapse of the support structure of the distal phalanx and in larger individuals the distal phalanx protrudes through the sole of the foot. When the connection between the sensitive laminae and non sensitive laminae weakens, the coffin bone can rotate and/or sink. The syndrome can proceed through several stages. At the one end of the scale is mild inflammation which can be treated, with little permanent damage. This scenario is uncommon. Most cases of laminitis at the other end of the scale lead to chronic conditions and total loss of use or reduced use. This condition is irreversible and extremely painful and euthanasia is indicated.

Recapitulating the definition of laminitis is: the failure of the inner hoof wall to attach to the bone inside the hoof, called the coffin bone. The hoof wall is held in place by a special type of folded tissue called laminae. Laminitis causes inflammation of this tissue and may lead to such destruction inflammation of this tissue and may lead to such destruction of the laminae that the hoof wall becomes detached from the coffin bone which sets inside of it. The coffin bone “sinks,” causing much pain to the horse.

Without proper attachment to the inner hoof wall, the weight of the horse and the forces of movement drive the coffin bone down, damaging arteries and veins and crushing the living tissues of the coronary band and sole. This leads to unrelenting pain and a characteristic lameness.

3. Description of the Related Art

In the past, treatment was limited to physical treatment of the affected foot, and more particularly to an apparatus which is placed between a horse's hoof and a horseshoe to properly reposition the rotated out of position coffin bone of a horse. An Example of such prior art references includes U.S. Pat. No. 4,794,991.

Currently these conditions are treated in a variety of ways: either through the use of pharmacological agents or surgery, or a combination of both. Chemical treatment of symptoms in the manner described are known from U.S. Pat. Nos. 6,534,526 and 6,045,827.

There are many techniques for helping these patients as mentioned above but the expense of continual treatment in terms of medicines, shoeing cannot be sustained by many owners especially in the face of reduced performance.

In addition all pharmacological agents have a degree of complication associated with them.

Surgery brings all the risks of anesthesia or sedation and the potential complications of any surgical procedure

The present invention is related to a method for treating laminitis in Horses, Ponies and other Equines with acoustic shock waves.

### BRIEF SUMMARY OF THE INVENTION

In general, the present invention is directed to a method for treating laminitis in animals. More specifically, the present invention relates to a method for veterinary medical treatment of Laminitis in Horses, Ponies and other Equines with a sufficient number of acoustic shock waves. The novel method described herein uses acoustic shock waves to normalize the blood flow within the laminae and thereby reverse the laminitic process.

The current state of the art technology for producing extra corporeal shock waves in the most appropriate clinical manner is described below.

Shock waves are high-energy acoustic waves with high amplitude, characterized by extremely short build-up times. The shock waves are generated outside the body and can be focused at a specific site within the body.

The most common use of shock waves in humans is to break kidney and urethral stones into small fragments which can then be eliminated from the body. This is known as lithotripsy. In addition shock waves are now routinely used to treat common orthopedic problems in humans such as tennis elbow and non-healing fractures.

The method of treatment of the present invention includes applying a sufficient number of acoustic shock waves to the side of a abnormal condition to induce or accelerate healing. Applying the acoustic shock waves involves the deliberate introduction of a series of very high pressure, focused or non focused pulses into the area to be treated. The shock waves travel through fluid and soft tissue with their effects occurring at places where there is a change in the type of tissue. High forces of compression and tension are created as these acoustic shock waves meet a different type of tissue.

Applying the acoustic shock waves involves generating preferably about 500 to about 4000 acoustic shock waves. The acoustic shock waves may be applied either in a single treatment or in multiple treatments.

The application of shock waves to the equine foot using a shock wave generator would be an effective treatment in laminitis which through normalizing the compartment syndrome in the foot would prevent permanent damage to the lamina. Thereby reducing pain and permanent disability.

This novel method of application of shock waves reduces the number of re-treatments and produces therapeutic healing of these conditions with minimal risk of complications or damage. Generally speaking, it is hypothesized that shock waves triggers physiological repair mechanisms.

Furthermore, it has been observed to produce marked improvements in conditions which previously had no realistic hope of resolution, even with severe surgical measures with all the attendant risks. Surgery brings all the risk of anesthesia or sedation and the potential complication of any surgical procedure, including sepsis and continued morbidity.

The inventional method for treating laminitis diseases bears far less risk to the patient than pharmacological and surgical intervention.

### BRIEF DESCRIPTION OF THE DRAWING

These and other features, aspects and advantages of the present invention will become better understood with reference to the following description, appended claims, and accompanying drawing, where:

FIG. 1 is a schematic representation of a shock wave generation device using the electro-hydraulic principle in accordance with the inventive method and

FIG. 2 is a schematic representation of a shock-wave generator and positioning in accordance with the inventive method.

In the drawing, the principle of producing the shock waves is depicted only schematically. Equivalent parts are indicated by the same reference numbers.

List of Reference Numbers

2 shock wave generation device

4 electrode

6 focusing device

8 propagation of a high-pressure shock waves

10 focal peak pressure area

12 coffin bone

14 foot (lateral view)

Equivalent parts are indicated by the same reference numbers.

### DETAILED DESCRIPTION OF THE INVENTION

The invention summarized above and defined by the enumerated claims may be better understood by referring to the following detailed description, which should be read in conjunction with the accompanying drawing. This detailed description of a particular preferred embodiment, set out below to enable one to practice the invention, is not intended to limit the enumerated claims, but to serve as a particular example thereof.

The present invention is generally directed to treatment of laminitis in hoofed animals. In general, the objects and advantages of the present invention are achieved by applying to a laminitic animal acoustic shock waves.

Implementation of the method of the present invention requires the use of a shock wave source.

Currently there are three distinct methods of acoustic shock wave generation in medicine; the piezoelectric method, the electromagnetic method and the electro hydraulic method.

The preferential method of shock wave generation for the inventional treatment of laminitis in horses, ponies and other equines is the electro hydraulic method. But the novel invention is not limited to the electro hydraulic method of shock wave generation only.

The reason for utilizing the electro hydraulic method of shock wave generation is, that this method enables both the generation of high-pressure shock waves and of a large focal volume.

Specifically, for the electro hydraulic method the inventive method may include applying a range of approximately 14-28 kilovolts of energy pulse.

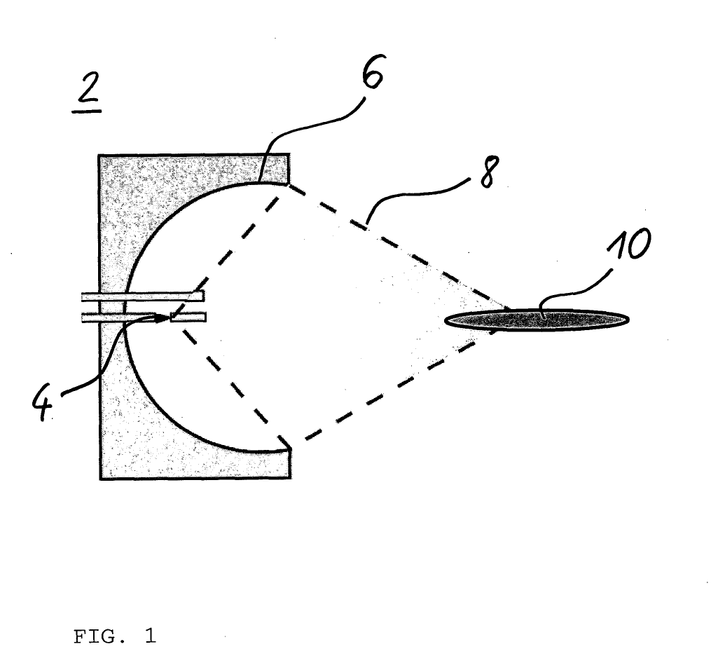

FIG. 1 illustrates a schematic of a shock wave generation device 2 with the electro hydraulic principle. Capacitors, not shown in the schematic, are charged with high voltage. The shock wave is generated by discharging the capacitors across the tips of an electrode 4 mounted in a focusing apparatus 6 preferably a fluid filled ellipsoid reflector. The subsequent, sudden vaporization of the water molecules between the tips of the electrode 4 causes the spherical propagation 8 of a high-pressure shock wave through the surrounding fluid. The method typically requires use of apparatus for focusing 6 the acoustic shock waves, such as an ellipsoid or parabolic reflector or focusing lens. The reflector 6 is generally located in the shock wave generation device 2, which directs the waves to a focal peak pressure area 10. Shock waves travel from electrode 4 through acoustic medium, which is not explicit shown in FIG. 1. Waves reflect from the ellipsoid surface of the reflector 6 and toward peak focal pressure area 10.

Due to the anatomical features of the equine foot the shock-wave generation device 2 needed would have to be such that the shock waves could be introduced below the horn. In a preferred embodiment, the shock wave generation device 2 is designed as a hand-held shock-wave generator. This is advantageous to deliver the shock waves to the animal in the appropriate fashion. In a further embodiment there are more than one hand-held shock-wave generator devices 2 available. Each of the hand-held shock-wave generator devices 2 is characterized by a specific depth, at which the focal peak pressure area 10 is located.

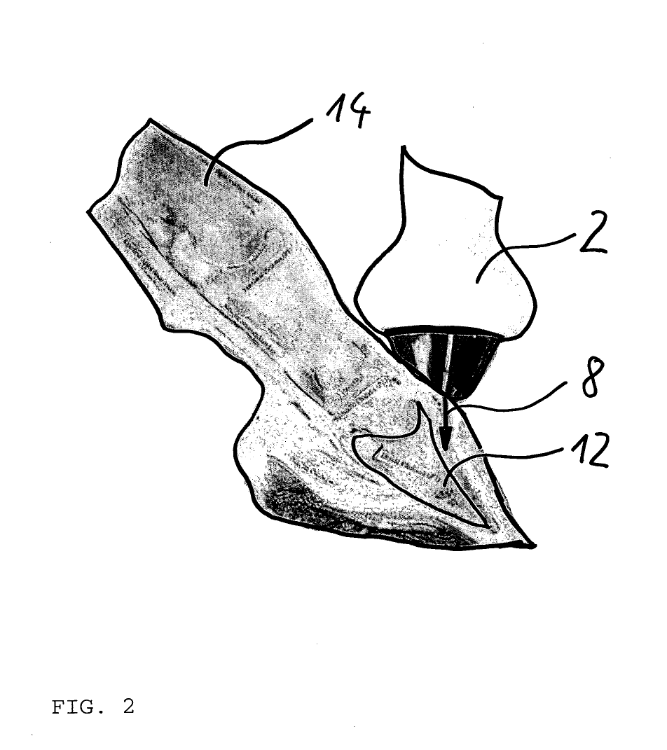

The novel method for treating laminitis in animals with shock waves described here and as is illustrated in FIG. 2 is to place the shock wave generation device 2 such that the shock wave passes between the pastern through the perioplic and coronary corium down into the laminar corium. This can be achieved all the way around the periople and between the heel bulbs.

In addition the novel method may utilize physical palpation, X-ray localization or ultrasound precisely to locate the site on which the shock waves have to be applied.

Applying the acoustic shock waves involves generating about 500 to about 4000 acoustic shock waves. The shock wave pulse frequency may be approximately 0.5-4 Hz (shock wave pulses/sec). Additionally, the number of treatments necessary for a positive response may vary from 1 to 4.

While the invention has been described in terms of various preferred embodiments, those of skill in the art will appreciate that various modifications, substitutions, omissions and changes may be made without departing from the spirit thereof. Accordingly, it is intended that the scope of the present invention not be limited solely by the scope of the claims.

What is claimed is:

1. A method for treating laminitis in horses, in particular ponies and other equines comprising:
locating a site or suspected site of an abnormal condition of laminitis;
generating acoustic shock waves;
introduction said acoustic shock waves on said located site; and
applying a sufficient number of said acoustic shock waves to said located site to induce or accelerate healing.

2. The method of claim 1, wherein said acoustic shock waves are focused acoustic shock waves.

3. The method of claim 1, wherein said acoustic shock waves are non focused acoustic shock waves.

4. The method of claim 1, wherein said shock waves pass between the pastern through the perioplic and coronary corium down into the laminar corium.

5. The method of claim 1, wherein applying a sufficient number of said acoustic shock wave comprises applying about 500 to about 4000 of said acoustic shock waves per treatment.

6. The method of claim 1, wherein applying a sufficient number of said acoustic shock waves comprises applying said acoustic shock waves in a single treatment.

7. The method of claim 1, wherein applying a sufficient number of said acoustic shock wave comprises applying said acoustic shock waves in a multiple treatment.

8. The method of claim 1, wherein the preferential method of shock wave generation is the electro hydraulic method.

9. The method of claim 8, wherein said electro hydraulic shock wave generating comprises generating said acoustic shock waves by applying a voltage potential across a spar gap generator ranging from about 14 kV to about 28 kV to generate each shock wave

10. The method of claim 1, wherein the method of shock wave generation is the piezoelectric method, the electromagnetic method or the pneumatic or ballistic method.
